(19) United States
(12) Patent Application Publication
(10) Pub. No.: US 2017/0136210 A1
(43) Pub. Date: May 18, 2017

(54) NON-FOULING, ANTI-MICROBIAL CATHETER

(71) Applicant: Puratec, LLC, Raleigh, NC (US)

(72) Inventors: Paul Robertson, Raleigh, NC (US); Edward Vresilovic, Ardmore, PA (US); Daniel A. Pontecorvo, Newtown Square, PA (US)

(21) Appl. No.: 15/349,633

(22) Filed: Nov. 11, 2016

Related U.S. Application Data
(60) Provisional application No. 62/256,541, filed on Nov. 17, 2015.

(51) Int. Cl. A61M 25/00

(52) U.S. Cl. CPC A61M 25/0043; A61M 25/0017

(57) ABSTRACT

The disclosed non-fouling, antimicrobial catheters release a therapeutically effective amount of silver ions in response to an electrical current to prevent, inhibit, or reduce bacteria or fungi from passing through the catheter into the body of a subject, for example passing through the urethra and into the bladder via the gap between the external surface of the catheter and the urethral wall. The release of silver ions in response to the electrical current also inhibits, reduces, or prevents microbes from passing through the catheter into the subject via the internal features of the catheter. The release of silver ions also reduces, inhibits or prevents microbes from colonizing and developing a fibrous matrix on the exterior surface of the catheter (also known as biofilm). Microbes on the catheter or in the tissue of a subject in contact with the catheter are killed by the ionic silver.

### CROSS-REFERENCE TO RELATED APPLICATIONS

This application claims benefit of U.S. Provisional Application No. 62/256,541 filed Nov. 17, 2015, which is incorporated by reference in its entirety.

### FIELD OF THE INVENTION

The invention is generally directed to catheters that release ionic silver, preferably in response to the application of electric current to the catheter.

### BACKGROUND OF THE INVENTION

The indwelling urinary catheter was introduced by F. E. B. Foley in 1927 (Foley, J Urol, 21:289-306 (1929)). Since its inception, the indwelling catheter has been associated with complications, some of which are severe. Today, indwelling urinary catheters are a leading cause of healthcare-associated urinary tract infections (UTIs) (van den Broek, P. J., et. al., (2011)). Symptoms of a UTI may include: fever, chills, headache, burning of the urethra or genital area, leaking of urine out of the catheter, blood in the urine, foul smelling urine, low back pain and achiness. Additional complications associated with urinary catheters include, but are not limited to allergic reaction to the material used in the catheter, such as latex, bladder stones, injury to the urethra, kidney damage (with long-term indwelling catheters), and infection of the urinary tract, kidney, or blood (septicemia). Urinary catheter associated bloodstream infection is a serious complication that can result in death (Bursle, E. C., J Infect., pii: S0163-4453 (2015)).

Other types of catheters can also be sources of infection. These catheters include vascular catheters such as central venous catheters (CVCs), peripherally inserted catheters and (PICCs), and endotracheal catheters. Regardless of the type of catheter, the catheter serves as an entry point for microbial infection.

A typical catheter-related urinary tract infection can occur, for example, when a urethral catheter inoculates organisms into the bladder and thereby promotes colonization by providing a surface for bacterial adhesion and causing mucosal irritation. (Vergidis, P, and Patel, R. Infect Dis Clin North Am, 26(1):173-86 (2012)). The presence of a urinary catheter is the most important risk factor for bacteriuria.

Once a catheter is placed, the daily incidence of bacteriuria is 3-10%. Between 10% and 30% of patients who undergo short-term catheterization (i.e., 2-4 days) develop bacteriuria and are asymptomatic. Between 90% and 100% of patients who undergo long-term catheterization develop bacteriuria. About 80% of nosocomial UTIs are related to urethral catheterization; only 5-10% are related to genitourinary manipulation. (John L Brusch, Medscape, 2013).

Exemplary pathogens include Escherichia coli, Pseudomonas species, Enterococcus species, Staphylococcus aureus, coagulase-negative staphylococci, Enterobacter species, and yeast. Proteus and Pseudomonas species are the organisms most commonly associated with biofilm growth on catheters. More recently, anti-biotic resistant strains of bacteria are becoming problematic.

Thus, there is a need for antimicrobial catheters that reduce or inhibit microbial infections in a subject.

It is an object of the present invention to provide methods and catheters to reduce, inhibit, or prevent catheter-related microbial infections.

It is another object of the invention to provide a non-fouling, antimicrobial catheter.

It is another object to provide an antimicrobial catheter that releases an effective amount of an antimicrobial metal to inhibit, reduce or prevent biofilm formation on the catheter.

### SUMMARY OF THE INVENTION

The disclosed non-fouling, antimicrobial catheters release a therapeutically effective amount of silver ions in response to an electrical current to prevent, inhibit, or reduce bacteria or fungi from passing through the catheter into the body of a subject, for example passing through the urethra and into the bladder via the gap between the external surface of the catheter and the urethral wall. The release of silver ions in response to the electrical current also inhibits, reduces, or prevents microbes from passing through the catheter into the subject via the internal features of the catheter. The release of silver ions also reduces, inhibits or prevents microbes from colonizing and developing a fibrous matrix on the exterior surface of the catheter (also known as biofilm). Microbes on the catheter, in the urine, or in the tissue of a subject in contact with the catheter are killed by the ionic silver.

The catheter can be a Foley catheter (indwelling catheter), venous catheter (CVCs), peripherally inserted catheter (PICCs), or an endotracheal catheter. It has been discovered that changing the polarity of the current as well as modulating the amount of current enables the disclosed catheters to release therapeutically effective amounts of the silver ions over prolonged periods of time. For example, some embodiment provides catheters that alternated current from 0.0 μAmps to 100 μAmps, preferably between 20 μAmps and 60 μAmps. In one embodiment, the current is alternated from −150 μAmps to +150, preferably about −60 μAmps to +60 μAmps, or even −20 μAmps to +20 μAmps. In one embodiment, the catheter releases a therapeutic amount of silver ions for at least 7 days without complications including infections.

In one embodiment, the disclosed catheters are urinary catheters used for draining the bladder.

### BRIEF DESCRIPTION OF THE DRAWINGS

FIG. 1 is a side view of an exemplary catheter with axial electrodes.

FIG. 2 is a side view of an exemplary catheter with printed axial electrodes.

FIG. 3 is a side view of an exemplary catheter with helical printed electrodes including a solid silver “flat wire”.

FIG. 4 is a side view of an exemplary catheter with bookend ring electrodes.

FIG. 5 is a side view of an exemplary catheter with a common cathode ring electrode.

FIG. 6 is a side view of an exemplary battery configuration for an exemplary catheter.

FIG. 7 is a side view of an exemplary battery configuration with the electrical connector tethered to a power source.

FIG. 8A is a side view of an exemplary circular housing for electronics of an exemplary catheter.

FIG. 8B is a top cross sectional view of the configuration of FIG. 8A.

FIG. 9A is a side view of an exemplary rectangular housing for electronics of an exemplary catheter. FIG. 9B is a side view of the catheter configuration of FIG. 9A.

FIGS. 10A-10F show several views of another embodiment of the catheter containing an impedance element. FIG. 10A is a broken side view. FIG. 10B is broken top view. FIG. 10C is a cross-sectional view of the shaft taken at position A. FIG. 10D is a cross-sectional view of the shaft taken at position B. FIG. 10E is a cross-sectional view of the shaft taken at position C. FIG. 10F is a cross-sectional view of the shaft taken at position D.

FIGS. 11A and 11B are conceptual renderings illustrating the impedance across the insulator.

### DETAILED DESCRIPTION OF THE INVENTION

### I. Definitions

The term “therapeutic amounts of ionic silver” refers to amounts of ionic silver that inhibit, reduce, or prevent microbial infections resulting from bacteria or fungi including antibiotic resistant microorganisms. The term “therapeutic amounts of ionic silver” refers to more than antimicrobial activity of a few microorganisms. As used herein the term refers to the amount of ionic silver needed to prevent or inhibit the geometric growth of the microorganisms and/or the amount of ionic silver necessary to inhibit or prevent the formation of biofilm. Typically, the amount of ionic silver is expressed as a concentration of ionic silver that is maintained over a period of time. For example, a therapeutic amount of ionic silver includes, but is not limited to greater than 5 μg/ml to 100 μg/ml of ionic silver for at least 2 hours, preferably for 1 to 7 days.

The term “Foley catheter” refers to an indwelling catheter, typically an indwelling urinary catheter.

The term “antibiotic resistant microorganisms” refers to and includes but is not limited to Clostridium difficile (CDIFF carbapenem-resistant Enterobacteriaceae (CRE), drug resistant Neisseria gonorrhoeae, multidrug-resistant Acinetobacter, drug-resistant Campylobacter, fluconazole-resistant Candida, Extended Spectrum Enterobacteriaceae (ESBL), vancomycin-resistant Enterococcus (VRE), multidrug-resistant Pseudomonas aeruginosa, drug-resistant non-typhoidal Salmonella, drug-resistant Salmonella Serotype Typhi, drug-resistant Shigella collapsed, methicillin-resistant Staphylococcus aureus (MRSA), drug-resistant Streptococcus pneumoniae, drug-resistant tuberculosis, vancomycin-resistant Staphylococcus aureus, erythromycin-resistant Group A Streptococcus, and clindamycin-resistant Group B Streptococcus. The disclosed catheters preferably release amounts of ionic silver to inhibit, reduce, or prevent infection from at least one antibiotic resistant microorganism, preferably at least two antibiotic resistant microorganisms, and most preferably at least three antibiotic resistant microorganisms.

The term “biofilm” refers to an assemblage of surface-associated microbial cells that is enclosed in an extracellular polymeric substance matrix. (R. Donlan, Emerging Infectious Diseases, 8(9):881-890 (2002)). Microorganisms commonly associated with biofilms on indwelling medical devices include, but are not limited to Candida albicans, Coagulase-negative staphylococci, Enterococcus spp., Klebsiella pneumonia, Pseudomonas aeruginosa, and Staphylococcus aureus.

The term “non-fouling” refers to a surface or instrument that resists or inhibits the formation of a biofilm.

### II. Non-Fouling, Antimicrobial Catheters

### A. Core Components

The disclosed catheters share core components with conventional catheters. A conventional catheter is typically a flexible tube that is inserted in the body. Urinary catheters are flexible tubes that go through the urethra and into the bladder to drain urine out of the bladder. A typical urinary catheter has two separated channels, or lumens, running down its length. One lumen is open at both ends, and allows urine to drain out into a collection bag and typically has a funnel connector. The other lumen has a valve on the outside end and connects to a balloon at the tip. The balloon is inflated with sterile water when inside the bladder to prevent it from accidently slipping out of the bladder. The balloon lumen can have a one-way luer activated valve for syringes.

The balloon component can withstand external pressure in bladder without deforming to a point of allowing sliding back through urethra. Additionally, the balloon component can withstand internal pressures to allow safe inflation with saline. The tubing can be made of materials such as silicone or polyurethane, latex, or polyvinyl chloride (PVC). The diameter of the tubing range from 6 to 30 F, with pediatrics approx. 6-12 F. The relative size of a catheter is described using French units (F) . . . 1 F is equivalent to 0.33 mm=0.013″= 1/77″ of diameter. 14-16-18 F appear to be the most common. Typical Foley catheter shaft lengths are approximately 32-35 cm (13-14 inch). Intermittent urinary catheters have male (16″ OAL) and female (6″ OAL) specific offerings and therefore have shorter length versions. Exemplary catheters have a size of 16 F (approximately 0.210″ shaft diameter) and length 33 CM.

### B. Electrodes

The electrodes are preferably silver or a silver alloy capable of releasing silver ions in response to electric current. In certain embodiments, the electrodes are contain enough silver metal to release therapeutic amounts of silver in response to current passed through the electrodes. In other embodiments, the electrodes are in contact with silver metal on the electrode so that when electricity is passed through the electrodes, the silver metal releases a therapeutic amount of ionic silver.

In one embodiment the electrode is placed on the exterior surface of the catheter and is constructed to provide an electrically active field in a strategically placed zone that maximizes the desired antimicrobial effect. One embodiment provides the electrodes inside the bladder outlet (conical section of bladder), therefore in the location just proximal to the balloon, or approx. 5-7 cm from the distal tip of the catheter.

Another embodiment has the electrodes placed inside the urethra, close to the urethral opening: approx. 9-12 cm from the distal tip for an average adult female, 24-27 cm for average male.

Still another embodiment provides the electrodes inside the bladder, therefore located on the most distal 1-2 cm of the catheter shaft.

One embodiment has a distinct electrode pairs running axially for approximately 20-40 mm and 180 degrees from each other around the catheter shaft.

Another embodiment has continuous electrode “stripes” with length approximately 40 mm and spaced 180 deg from each other. The number of electrode stripes can vary. For example, the electrode can have four anode and for cathode stripes. The electrode stripes can be produced using “silver ink” or can be solid wires. One type of conductive ink that may be used is a silver-based epoxy ink. Conductive ink of this type may be obtained from Creative Metals Incorporated as product number CMI 119-21 and referred to as Printable Solvent Resistant Electrically Inductive Ink.

Another embodiment provides a continuous electrode helix, with length approximately 40 mm of the catheter shaft.

Another embodiment provides electrode rings around the shaft (360 degree), for example a pair of rings spaced axially a distance 40 mm from each other.

Yet another embodiment provides a catheter with electrode rings sharing a common cathode, for example two anodes with the common cathode.

### C. Electronics

The disclosed catheters include electronics for controlling the amount of current passing through the electrodes as well as for alternating the polarity of the electric current. The electronics are attached to the catheter in such a way that it does not encumber the end user. The location of electronics can be integrated with a juncture/manifold hub. See FIGS. 6, 8A, 9A and 9B.

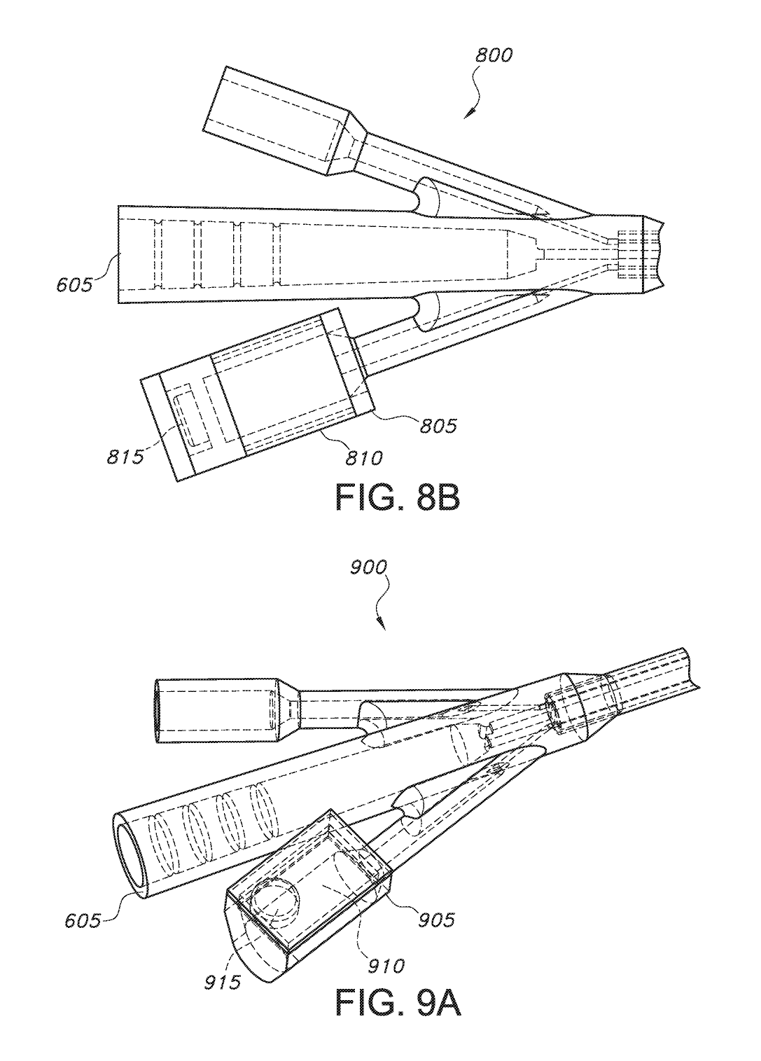

The electronics can be primarily contained in a small envelop or housing. In FIG. 8, the diameter of the housing is approx. 0.5 inch and the length is approx. 1.0 inch.

The electronic board shall include a power source, i.e., a battery (or batteries), capable of being programmed to deliver a range of constant currents. Likely current range is between 20 and 150 μA for at least one, two, three, four or as many as 20 weeks of continuous operation. An exemplary battery is SR66, a commercially available super silver oxide 1.5V battery.

In one embodiment, the electronics is capable of delivering a range of current density from approximately 1-5 A/m², which is dependent upon the programmed current and electrodes' configuration.

The electronics preferably to self-adjust power consumption to be of a constant current type. Additionally, the electronics is capable of alternating and cycling the current from positive to negative and back to positive (i.e., swapping poles)

In one embodiment the electronics can be programmed to deliver a predefined duty cycle that includes at least 2 current magnitudes, directions, and durations, preferably 4 and can have 1-8 cycle periods. For example, a typical cycle can be:

### 1. +60 μA for 1 min

### 2. all off (0 μA) for 2 min

### 3. −60 μA for 1 min

### 4. all off (0 μA) for 2 min

5. Back to step 1

| | Example 1 | | Example 2 | | Example 3 | | Example 4 | | Example 5 | |
|---|---|---|---|---|---|---|---|---|---|---|
| Cycle period | +60 | 1 | +60 | 10 | +60 | 5 | +150 | 2 | +20 | 12 |
| 1 | μA | min. | μA | min. | μA | min. | μA | min. | μA | hours |
| Cycle period | 0 | 5 | −60 | 10 | 0 | 1 | +20 | 30 | −20 | 12 |
| 2 | μA | min. | μA | min. | μA | min. | μA | min. | μA | hours |
| Cycle period | −60 | 1 | +60 | 10 | −60 | 5 | −20 | 30 | +150 | 1 |
| 3 | μA | min. | μA | min. | μA | min. | μA | min. | μA | min. |
| Cycle period | 0 | 5 | −60 | 10 | 0 | 1 | 0 | 1 | −150 | 1 |
| 4 | μA | min. | μA | min. | μA | min. | μA | hour | μA | min. |

The activation of the power can be “automatic” in that current will flow when the circuit is completed via body fluids. Alternatively, fluids could be provided on the catheter to activate the electrodes. This could be of the form of a saline wash running through micro channels of the catheter, or otherwise an aqueous sublayer

Additional optional features that should be considered include an ON/OFF switch, visual alarm when current is active (e.g., LED), visual alarm when ON, but not active (i.e., on but no current flow), and visual low battery alarm. Additional features include diagnostics such as communications external to the device, for collecting and storing current and/or voltage data, etc.

### D. Exemplary Embodiments

As noted above, the disclosed catheters can be any form of catheter.

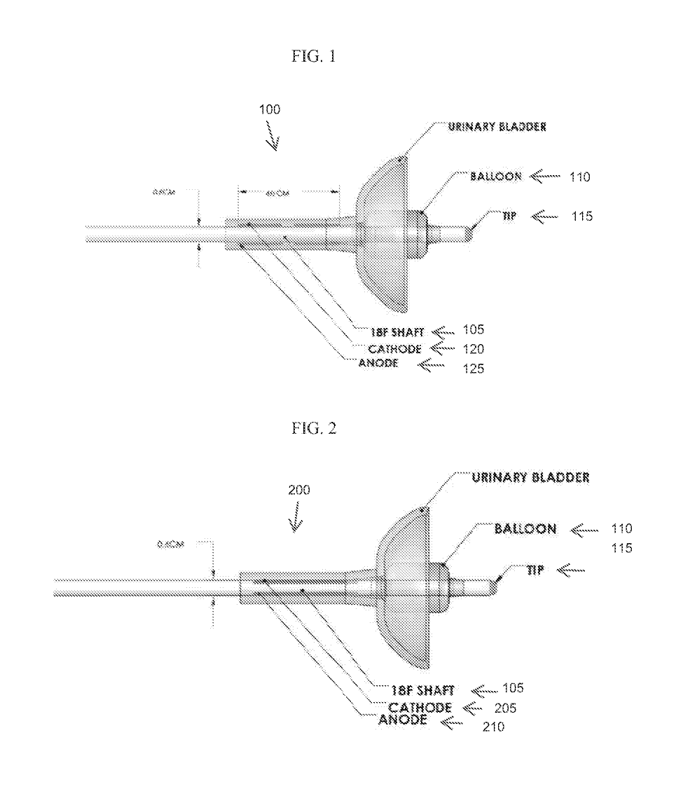

Preferably, the disclosed catheters are indwelling catheters such as urinary catheters. FIG. 1 shows an exemplary distal end of an indwelling catheter 100 containing an 18 F shaft 105. The proximal end is not shown. Exemplary proximal ends are shown in FIGS. 6-9B. Shaft 105 contains two separated channels, or lumens, running down its length. One lumen is open at both ends, and allows urine to drain out into a collection bag. The other lumen has a valve on the outside end and connects to a balloon 110 at the tip 115; the balloon 110 is inflated with sterile water when it lies inside the bladder 120, in order to stop the catheter 100 from slipping out of the bladder. Catheter 100 has an axial anode 125 and cathode 120. As shown, there are two anodes and two cathodes (or two pairs). It will be appreciated that the catheter could contain just one pair of electrodes. The two pairs of electrodes can be on the same circuit (in “series”, just with two separate exposures to the body) or the two pair of electrodes can be on separate circuits (in “parallel”). The latter has the advantage that ions can be released equally in two locations whereas the former, the possibility exists that the ions release mainly in one location because the current takes the easiest path.

In one embodiment the catheter has a third lumen that is dedicated to the electrical wires. There is a possibility that “the wires” are external such as the completely printed version (not shown) or the wires are completely embedded in the wall of the shaft and not down a lumen.

FIG. 2 shows another embodiment of the distal end of an indwelling catheter 200 having printed axial cathode 205 and printed axial anode 210. The printed electrodes are formed by using a conductive ink such as those available from Creative Metals Incorporated as product number CMI 119-21 and referred to as Printable Solvent Resistant Electrically Inductive Ink. Alternatively, the electrodes can be made of pure silver wire (99.9% pure).

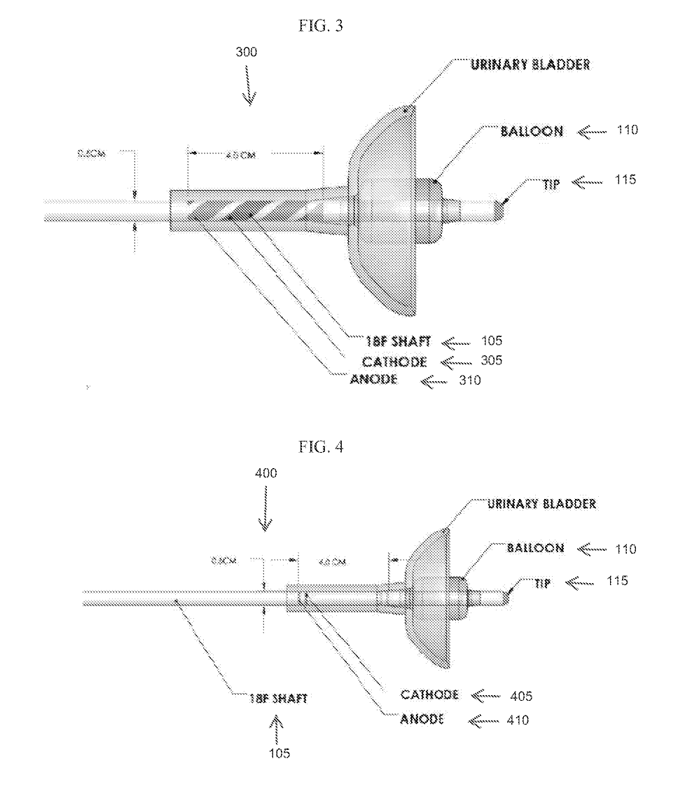

FIG. 3 shows another embodiment of the distal end of an indwelling catheter 300 (proximal end not shown). In this configuration the cathode 305 and anode 310 are printed onto the catheter in a helical pattern or made of pure silver wire. The wire can be round or flat.

FIG. 4 shows still another embodiment of the distal end an indwelling catheter in which cathode 405 and anode 410 are book end ring electrodes. Again the proximal end of the catheter is not shown.

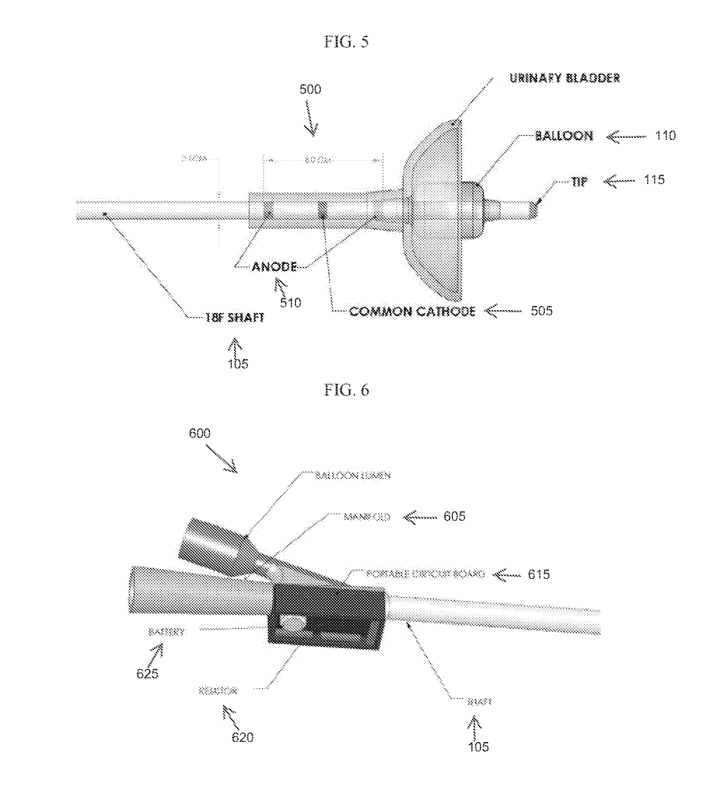

FIG. 5 is another embodiment of the distal end of an indwelling catheter 500 which contains a common cathode 505 for anodes 510. Again the proximal end of the catheter is not shown.

FIG. 6 is an embodiment of a proximal end 600 of an indwelling catheter containing a manifold 605 connected to shaft 105. A balloon lumen is distally also attached to shaft 105. Printed circuit board 615 contains a resistor 620 and a battery 625. Printed circuit board 615 can be programmed to provide specific duty cycles for the electrodes and specific amounts of current through the electrodes. In one embodiment, the duty cycle includes reversing the polarity of the electrodes for a defined amount of time.

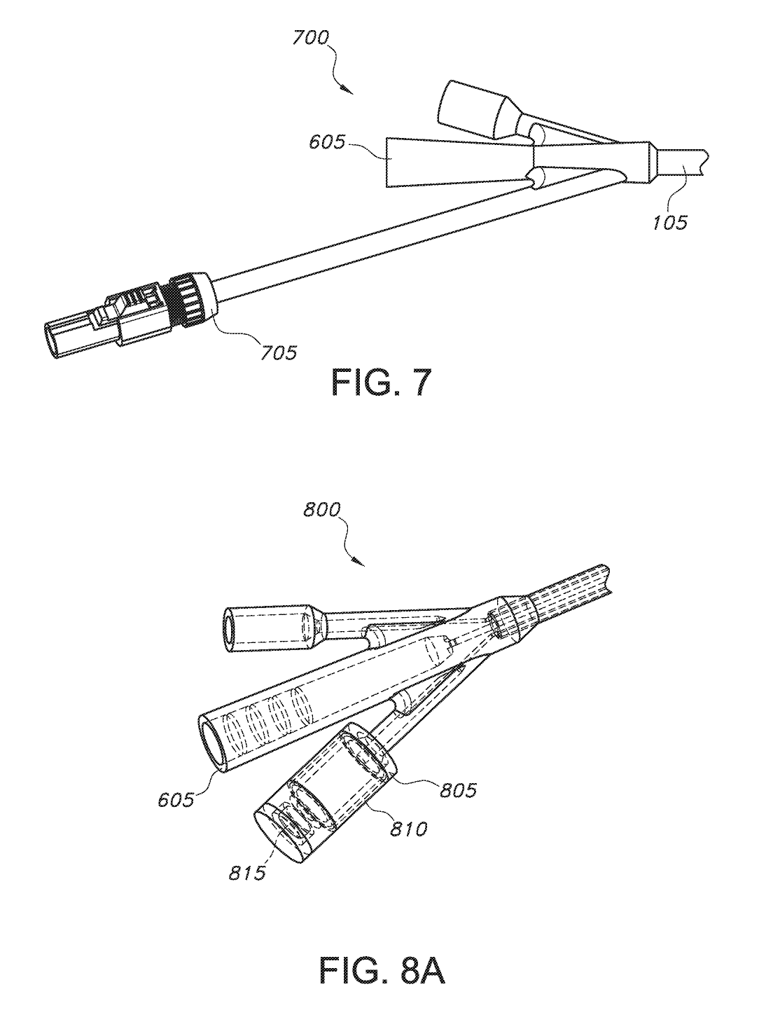

FIG. 7 is another embodiment of the proximal end 700 of an exemplary indwelling catheter. In this embodiment the proximal end 700 contains an electrical connector 705 tethered to a power pack 710.

FIG. 8A shows another embodiment of the proximal end 800 of an exemplary indwelling catheter. In this embodiment, proximal end 800 contains a circular electronic housing 805 which contains a flex circuit 810 and a battery 815. FIG. 8B is a top view of FIG. 8A.

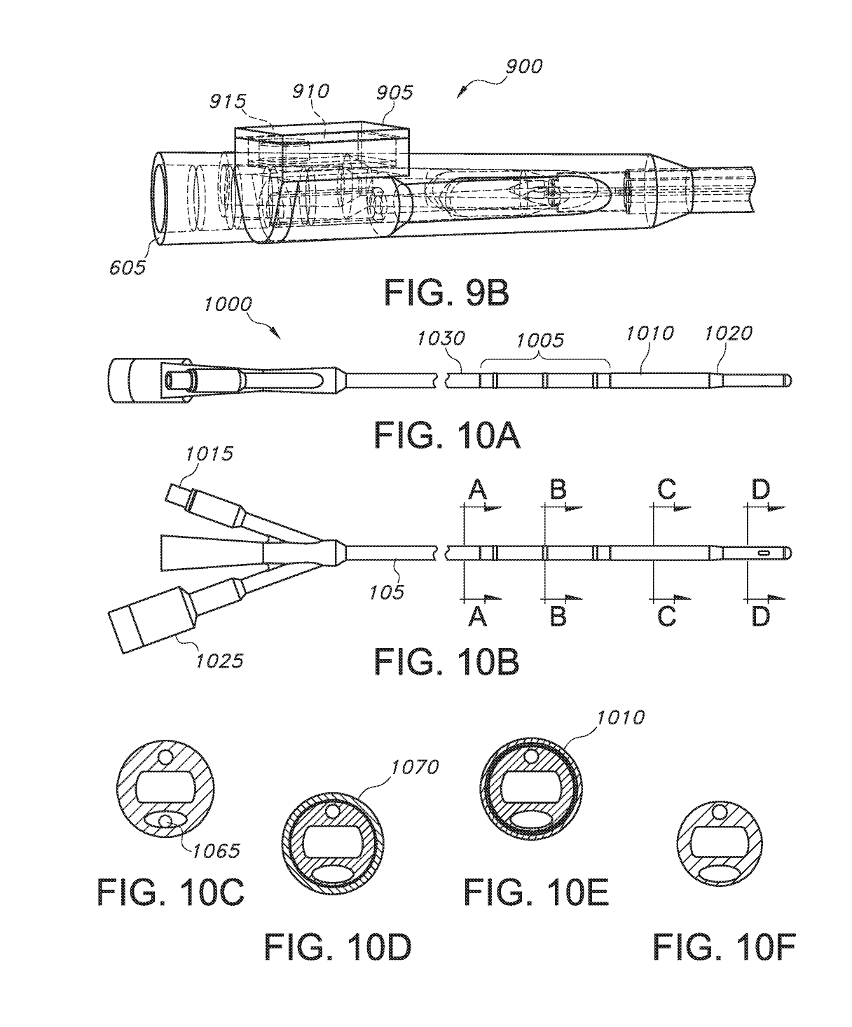

FIG. 9A shows another embodiment of the proximal end 900 for an exemplary indwelling catheter. This embodiment contains a rectangular electronics housing 905 which contains a circuit 910 and a battery 915. FIG. 9B is a side view of FIG. 9A.

FIGS. 10A-10F show another embodiment 1000 of the catheter containing an impedance element. This embodiment contains a ring electrode sleeve subassembly 1005. A silicon balloon is shown at 1010 followed by an adhesive fillet 1020. A 1-way luer activated valve is at 1015. The electronics subassembly 1025 is connected to the shaft 105. The 16 F to 14 F catheter subassembly is shown at 1030. FIG. 10C is a cross-section at position A of the shaft of the catheter 1000 showing a wire 1065 housed inside a lumen in the catheter. FIG. 10D is a cross-section at position B of the shaft of the catheter 1000 showing ring electrode 1070 surrounding the shaft. FIG. 10E is a cross-section at position C of the catheter 1000 showing balloon 1010 surrounding the shaft. FIG. 10F is a cross section at position D of the shaft of the catheter 1000.

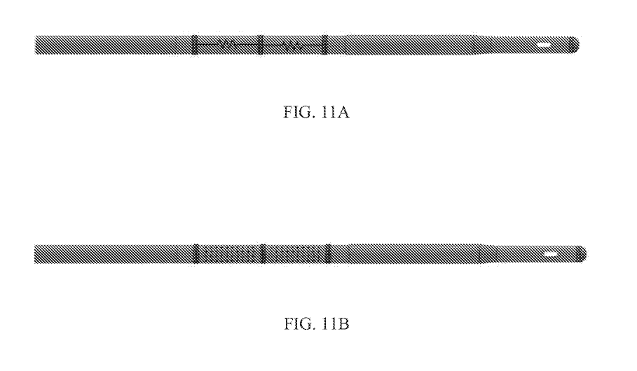

FIGS. 11A and 11B are conceptual renderings illustrating the impedance across the insulator.

One embodiment provides a catheter having a body made of a biocompatible material and comprising silver-containing electrodes and a power source having a first terminal and a second terminal with one of the terminals being in electrical communication with the silver-containing electrodes. The catheter also has an impedance device placed in a current path between the first terminal of the power source and the second terminal of the power source reducing or inhibiting current flowing from the first terminal from reaching the second terminal. Impedance devices are known in the art and include but are not limited to resistors. The impedance device can inhibit the current by 5, 10, 15, 20, 25, 30, 35, 40, 45, 50, 55, 60, 65, 70, 75, 80, 85, 90, 95, 99%. The catheter also includes a current generator connected to the power source that creates a flow of antimicrobial ions from the silver-containing electrode when current passes through the silver-containing electrode. The silver ions kill microbes in the environment of the catheter.

The catheter can also have electronics connected to the current generator to alternate cycling of the current from positive to negative and back to positive. In one embodiment, the current passes through along the silver-containing electrodes and the body tissue.

Another embodiment provides a method for inhibiting microbial infection associated with an indwelling catheter by providing a catheter as described herein. The method also includes delivering a current to the silver-containing electrodes when the catheter is located in a human body at a site of potential infection, and wherein the delivery of current to the silver-containing electrodes causes release of an effective amount of silver metal ions to inhibit the microbial infection. The infection can contain a Gram positive bacterium a Gram negative bacterium, a fungus or a combination thereof. In a preferred embodiment, the bacteria are resistant to antibiotics.

The current can be applied in a duty cycle that includes at least two current magnitudes, at least two current directions, and at least two current durations. For example, the current can be applied in a duty cycle that includes at least four current magnitudes, at least two current directions, and at least four current durations. In one embodiment, the current is applied in 1-8 cycle periods. In another embodiment, the cycle is:
a) +60 μA for 1 min
b) all off (0 μA) for 2 min
c) −60 μA for 1 min
d) all off (0 μA) for 2 min.
   The cycle can be repeated as many times as necessary to treat the microbial infection.

### EXAMPLES

### Example 1: Characterization of Catheters

To record the current across electrode catheters when the catheter's electrodes are submerged in 0.9% NaCl solution (NS) or 0.09% solution over a specified time period. This report contains a series of studies at various setup conditions, which are summarized in the Table 1.

Materials and Methods

Sample Preparation and Testing Procedure

Normal saline was prepared by weighing 4.5 g NaCl per 500 ml de-ionized water. Approx 30 ml or 10 ml of NS was placed in a vial which in turn was placed in a 37° C. water bath. The catheter was introduced through the lid of the vial at T=0 at which time the data logger was started. The catheter was plugged into its power box seconds later. The initial current was verified to be approx 19 μA for the 20 μA power box and approximately 61 μA for the 60 μA power box, confirming “normal” expected operation.

Results

TABLE 1

| Test Nr. | Date | Description/objective | PN of test article | New? |
|---|---|---|---|---|
| 1 | 12-Jan | Two hour test | A339A SN3 | Y |
| 2 | 13-Jan | Fresh Saline Study | A339A SN3 | N |
| 3 | 15-Jan | Electrode cleaning with IPA | A339A SN3 | N |
| 4 | 16-Jan | Electrode cleaning with IPA, 2nd time | A339A SN3 | N |
| 5 | 16-Jan | Electrode cleaning with IPA and 400 grit sandpaper | A339A SN3 | N |
| 6 | 17-Jan | Electrode cleaning, IPA, x4 | A339A SN3 | N |
| 6B | 18-Jan | Power Supply #1 stability | n/a (shorted) | n/a |
| 7 | 20-Jan | New catheter, out-of-box | A339A SN4 | Y |
| 8 | 20-Jan | New catheter, alternate electrode material | A339B SN11 | Y |
| 9 | 21-Jan | New catheter, axial stripe version | A347B SN18 | Y |
| 10 | 23-Jan | Electrode cleaning with IPA and TSB | A347B SN18 | N |
| 11 | 24-Jan | New axial electrode catheter in TSB | A347C SN13 | Y |
| 12 | 29-Jan | New ring catheter, wrapped silver wire on anode | A339A SN5 | Y |
| 13 | 11 Apr. 2014 | New 1-impression axial striped cath, 60 μA | A347A SN23 | Y |
| 14 | 14 Apr. 2014 | New 6-impression axial striped cath, 60 μA | A347D SN34 | Y |
| 15 | 15 May 2014 | Silicone cath. prototype with exposed electrodes, .008″ | N/A | Y |
| 16 | 21 May 2014 | Hanging wire surface area study, .008″ dia wire | N/A | Y |
| 17 | 28 May 2014 | Silicone catheter with silver ferrule | N/A | Y |
| 18 | 17 Jun. 2014 | Silicone catheter with exposed .020″ wire electrodes | A425 | Y |
| 19 | 17 Jun. 2014 | Silicone catheter with epoxy filled lumens, and wire | A425B | Y |
| | | elect. | | |
| 20 | 2 Jul. 2014 | Hanging wire area versus volume study | N/A | Y |
| 21 | 2 Jul. 2014 | Hanging epoxy test | N/A | Y |
| 22 | 7 Jul. 2014 | 5 mm Hanging wire, swapping poles avery 2 hrs. | N/A | Y |
| 23 | 4 Aug. 2014 | Silicone catheter with wire elect., >72 hrs | A425 | Y |
| 24 | 1 Sep. 2014 | Silicone catheter with 10 mm exposed wire electrode | A425 | Y |
| 25 | 8 Sep. 2014 | Hanging wire in TSB (and saline, switched poles . . .) | .050″ hang. wire | Y |
| 26 | 26 Sep. 2014 | HW with duty cycle 10-0-10-0 | .008 HW | Y |
| 27 | 30 Sep. 2014 | HW with duty cycle 5-5-5-5 | .008″ HW | Y |

The “New?” column indicates whether the catheter is new and untested (Y), or used and previously tested (N).
Other notable variables are listed in the results and/or on the graph attachment.

### Test No. 1

Detailed graphical results of Test No. 1 are attached. The catheter was designed with book end rings as shown in FIG. 4. The test was stopped after approximately 2 hours because of the instability observed. The graph indicates a steep drop in current after approx. 20 minutes. The catheter was examined under a microscope during the test, revealing a discoloration. Some oxidation appeared to be present on the positive electrode, i.e. there is browning on the left electrode compared to the right.

### Test No. 3

Cleaning of the electrodes from Test No. 1 and 2 with 70/30 isopropyl alcohol (“IPA”) sprayed onto a paper-towel resulted in the apparent removal of the residue and also smoothing of the electrode surface. Water bath was approximately 19° C. during test.

### Test No. 4

A second cleaning of the electrodes from Test No. 3 with IPA was less visually pleasing. There was obvious residual brown streaks that were not removable with normal cleaning. A lint-free cloth was used to clean the electrodes prior to the use of a paper towel.

### Test No. 5

A third cleaning of the electrodes from Test No. 4 with IPA along with more aggressive cleaning with 400 grit sand paper was performed. The electrode was refreshed in 0.9% saline. Although most of the oxidation appeared to be removed (or mixed in the surface), results were highly variable.

### Test No. 6

Electrodes were cleaned with IPA (4×) and the catheter was tested in the same saline vial (×2).

### Power Supply Stability Test

See attachment 6B.

### Test No. 7

A new catheter with book end ring electrodes was tested in fresh 0.9% saline.

### Test No. 8

A new catheter having book end ring electrodes with alternate electrode epoxy was tested in fresh 0.9% saline. Note that the graph's X axis is in hours. Much better results in terms of longevity of current were obtained. Oxidation could only be removed with a sharp object (scalpel).

### Test No. 9

A new catheter with axial stripe electrodes (FIG. 2) was tested in fresh 0.9% saline. Current was steady after 24 hours with minimal drop or fluctuation of current during the period.

### Test No. 10

The same catheter was cleaned with IPA which caused the removal of both the oxidation and the electrode material. The catheter was tested in attempt to use the proximal “good” section of the electrodes in approximately 45 ml of tryptic soy broth (“TSB”).

### Test No. 11

A new axial stripe electrode catheter was tested in TBS for 40 hour test.

### Test No. 12

A silver wire wrapped electrode was tested in TBS. The electrode was switched to 0.9% NaCl after approximate 3.5 days. The test ran for about 4.6 days.

### Test No. 13

A printed axial striped catheter was tested. One time impression (i.e., “thinnest” silver thickness) and with 60 μA. The test ran for about 3.5 before erratic behavior, with spikes and valleys averaging around 20 μA. The current recovery to 60 μA observed in the graph after 8 hrs. is the copper anode exposure. The silver based top coat deteriorated over time to the point where the electrical connectivity was made with the underlying copper wire, rebounding the current.

### Test No. 14

A printed axial striped catheter with 6× printed impressions (i.e., “thickest” silver thickness) was tested with 60 μA. The test ran for about 8 hrs before erratic behavior started. These catheters had 6 of 8 legs electrically active (2 were missing due to printing difficulty) and the poles were reversed to avoid a copper anode (underlying wire) connection. Contrasting the results with test Nr 13, areas of discontinuity can be seen visually on the parts, but with the darker corrosion starting proximal to distal, and a more “gradient” and less “abrupt” transitions to lighter corrosion. Some electrical conductivity occurred at the lid-line.

### Test No. 15

A marketed silicone catheter was modified with a 99.9% Ag ø.008″ wire and ø.020 Cu wire, and with 2 zones of exposure, totaling approx. 8 mm in length for each electrode.

### Test No. 16

Several hanging wire test samples were constructed with the intent of exposing progressively more anode surface area to saline to develop a baseline current versus surface area profile. Anode exposure to saline were 2, 3, 4, 5, 6, 7, 8, 9, 10 mm length of ø.008″ Ag wire. A ø.020″ Cu cathode was also used.

Approximate areas and volumes of the electrode are as follows:

| Length (mm) | total SA (mm\hat{ }2) | Volume (mm\hat{ }3) |
|---|---|---|
| 2 | 1.309 | 0.065 |
| 3 | 1.948 | 0.097 |
| 4 | 2.586 | 0.130 |
| 5 | 3.224 | 0.162 |
| 6 | 3.863 | 0.195 |
| 7 | 4.501 | 0.227 |
| 8 | 5.139 | 0.259 |
| 9 | 5.778 | 0.292 |
| 10 | 6.416 | 0.324 |

All current profiles were observed to have a similar pattern of a period of steady decline followed by a steep drop in current. Other notables are an increase in wire diameter after the test and what appears to be “coring” on the inside of the wire.

### Test No. 17

Test 17 shows a prototype construction whereby the anode electrode was created by drilling a ø0.050″ by 4 mm long Ag wire with a ø0.030″ hole (the ferrule) and threading a ø.016″ copper wire through the center and crimping the ferrule. The cathode was the same copper wire. The current stability graph reflects this.

### Test No. 18

Test 18 consisted of prototype A425, a silicone catheter with two Ag wires, each ø.020″, running down the shaft in separate lumens. The electrodes were created by exposing approx. 10 mm of wire length on each side. Both sides of the wire exposure was potted with silicone to prevent saline ingress to the lumens. This is the same prototype construction that was challenged with bacteria for 7.5 hrs. in test TR0070. The current stability test was run for 7 days.

### Test No. 19

A silicone catheter prototype was constructed by potting each side lumen with conductive 2-part epoxy, and “burying” a copper wire into the epoxy for a distance of approx. 300 mm on the proximal end of the catheter to make a connection to the power box cable, and burying a ø.020″×30 mm Ag wire to create the electrodes on the distal end (10 mm buried in epoxy, 10 mm exposed to surface, and another 10 mm buried in epoxy). The construction resulted in a desirable flexible shaft catheter; however the smaller dia. lumen did not have continuity, whereas the larger lumen did. Therefore, the current stability test was setup as a single pole catheter anode with a copper “hanging wire” cathode.

### Test No. 20

Several hanging wire test samples were constructed with the intent of exposing progressively more anode surface area or volume to saline to develop a baseline surface area versus volume profile. Anode exposure to saline were 2, 3, 4, 5, 6, 7, 8, 9, 10 mm length of ø.008″ Ag wire. To increase the surface area the same Ag wire lengths were flattened. A ø.020″ Cu cathode was also used.

Attachment 20 shows that the round anodes for all sizes (smaller surface) have a quicker decrease in current, but the overall time until a sudden drop is longer than the flat anodes (larger surface). For the flat anodes the decrease in current is slower but the sudden drop in current appears earlier than the round samples of the same exposed wire length. As expected the longer the anode (e.g. 10 mm versus 5 mm) the longer the period of steady decline until the steep drop in current.

### Test No. 21

A catheter prototype was created by the same methods as Test 19, but with no silver wire electrode attached. The goal of this study was to evaluate the silver-based conductive epoxy as an electrode. The anode (epoxy) was extracted from the catheter to be used in a hanging wire setup. Prior to the test, the epoxy was checked for electrical continuity, and it was confirmed very low and erratic. A scalpel was used to strip a layer from the top surface of the anode to improve continuity. It is likely that an electrical connection with the underlying copper wire was made after around 4 hrs.

### Test No. 22

Two sets of 5 mm hanging wire samples were energized with approx. 60 μA over a period of 14 hrs. One set maintained its current direction throughout the test, while the current of the second set alternated its direction every 2 hrs by swapping the catheter poles. The alternating set lasted longer with no steep decline after 14 hrs. It also appeared that, after the initial period, the current much is more stable when alternating, and becomes more steady over time.

### Test No. 23

A silicone catheter with silicone potted lumens, and wire electrodes was tested for >72 hrs. Test 23 consisted of prototype A425, a silicone catheter with two Ag wires, each ø.020″, running down the shaft in separate lumens. The electrodes were created by exposing approx. 10 mm of wire length on each side. Both sides of the wire exposure was potted with silicone to prevent saline ingress to the lumens. This is the same prototype construction that was challenged with bacteria for 7.5 hrs. in test TR0070. The current stability test was run for 5 days.

### Test No. 24

Test 24 used the previously mentioned prototype A425 with 10 mm silver wire exposed on two sides and potted lumens. The test was run for about 26 hours. This prototype was an untested catheter from the previous batch. The purpose of the test was to study the current stability of the catheter in TSB and 0.9% Saline. During the test, the catheter was switched alternatively between TSB and Saline. This test was a reaction to some poor current results during TR0074.

### Test No. 25

The main purpose of this test was to verify that current levels will be maintained beyond a 24 hr time period and in TSB solution, and in preparation for TP0087. Additionally and after 2 days, the poles were briefly swapped and the solution was changed to saline. These time points are indicated on the graph.

### Test No. 26

A duty cycle where electrodes were energized for 10 minutes (positive) before swapping the pole for 10 minutes (negative) was run for approx 24 hrs. (10-0-10-0 duty cycle). These HW Ag electrodes were ø0.008″×5 mm long. An identical electrode pair was run with continues current as a control.

Test No. 27-5 mm HW with 5-5-5-5 Duty Cycle

Test 27 was similar to Test No. 26 but with the introduction of “all offs” in the cycle (5-5-5-5 duty cycle). The assay was in a 0.09% saline solution. The test lasted about 3 days. The control electrodes lasted approximately 10-12 hrs, whereas the duty cycle lasted over 3 days with no signs of current degradation. The posts are insulated from each other the full length, with exception to the top of the screw within the insulator tubing.

### Example 2: Bacterial Testing

Materials and Methods

Results

| | | Test Type | Devices | Test | | |
|---|---|---|---|---|---|---|
| Expt | Title | Description | Tested | Duration | Pathogen | Results |
| TP0032 | Verification | Single point | Ring | 24 Hr | E. coli | No activity |
| | of | test for | electrode | | | |
| | Antimicrobial | adhered and | proto; | | | |
| | Effect of a | non- | Bardex | | | |
| | Catheter | adhered | | | | |
| | | pathogen | | | | |
| TR0042 | Current | Non- | Various | | Saline | Various |
| | Stability | biological | | | only | |
| | Testing | testing in | | | | |
| | | saline to | | | | |
| | | assess | | | | |
| | | current over | | | | |
| | | time | | | | |
| TP0043 | Verif. of | Zone of | N/A | N/A | N/A | N/A |
| | Antimicrobial | Inhibition | | | | |
| | Activity of a | test | | | | |
| | Catheter - Z | | | | | |
| | of I method | | | | | |
| TP0044 | Verif. of | Series of 6 | Bard | 2 Hr | E. coli | Minimal (1 |
| | Antimicrobial | pilot studies | catheter | | | log) |
| | Effect of a | to | pcs and | | | reduction |
| | Catheter - 2 | determine | printed | | | with |
| | hr test | growth | catheter | | | prototypes |
| | | curves | prototype | | | |
| TP0045 | Antimicrobial | Kill rate, | Copper | 7.5 Hr | E. coli | 8 log |
| R1 | Effectiveness, | Three test | and Ag | | | reduction |
| | Hanging | currents | wire | | | 60 uA, |
| | Wire in TBS | 0.5, 20, 60 | | | | 6 log |
| | | uA | | | | reduction |
| | | | | | | 20 uA |
| | | | | | | no |
| | | | | | | reduction at |
| | | | | | | 0.5 uA |
| TP0045 | Antimicrobial | Kill rate, | Copper | 7.5 Hr | S. aureus | 11-12 log |
| R1 | Effectiveness, | Three test | and Ag | | | reduction |
| | Hanging | currents | wire | | | 60 uA, |
| | Wire in TBS | 0.5, 20, 60 | | | | 11-12 log |
| | | uA | | | | reduction |
| | | | | | | 20 uA |
| | | | | | | no |
| | | | | | | reduction at |
| | | | | | | 0.5 uA |
| TP0045 | Antimicrobial | Kill rate, | Printed | 7.5 Hr | E. coli | 8 log |
| R1 | Effectiveness, | Two test | stripe | | | reduction |
| | Catheter in | currents | catheter | | | 60 uA, |
| | TBS | 20, 60 uA | prototype. | | | 8 log |
| | | | | | | reduction |
| | | | | | | 20 uA |
| TP0065 | Antimicrobial | Kill rate, | Printed | 7.5 Hr | E. coli | 9 log |
| | Effectiveness, | Two test | stripe | | | reduction |
| | Catheter in | currents | catheter | | | 60 uA, |
| | TBS | 20, 60 uA | prototype. | | | 9 log |
| | | | | | | reduction |
| | | | | | | 20 uA |
| TP0070 | Antimicrobial | Kill rate, | Silicone | 7.5 Hr | E. coli | 5 log |
| | Effectiveness, | Two test | proto | | | reduction |
| | Catheter in | currents | with Ag | | | 60 uA, |
| | TBS | 20, 60 uA | wires; | | | 8 log |
| | | | Bardex, | | | reduction |
| | | | Medline | | | 20 uA |
| TR0074 | Antimicrobial | Kill rate, | Silicone | 72 Hr | Klebsiella | Klebsiella |
| | Effectiveness, | Single test | proto | | Acinetobacter | No Change |
| | Catheter in | current 60 | with Ag | | Pseudomonas | Acinetobacter |
| | TBS | uA | wires; | | Enterococcus | 3 log |
| | | | Bardex | | | reduction |
| | | | | | | Pseudomonas |
| | | | | | | No Change |
| | | | | | | Enterococcus |
| | | | | | | 2 log |
| | | | | | | reduction |
| TP0087 | Antimicrobial | Kill rate, | Ag and | 7.5 Hr | E. coli | Ecoli |
| | Effectiveness | Single test | Ag wires | | Klebsiella | 8 log |
| | on | current 60 | | | Pseudomonas | reduction |
| | Superbugs, | uA | | | | Klebsiella |
| | Hanging | run in | | | | 5 log |
| | Wire in TSB | triplicate | | | | reduction |
| | | | | | | Pseudomonas |
| | | | | | | 8 log |
| | | | | | | reduction |
| TP0088 | Antimicrobial | Kill rate, | Ag and | 7.5 Hr | E. coli | DC 0.020 |
| | Effectiveness, | Single test | Ag wires | | | No Change |
| | Hanging | current 60 | | | | AC 0.020 |
| | Wire in TSB | uA with | | | | 6 log |
| | with Duty | Direct and | | | | reduction |
| | Cycle | Alternating | | | | DC 0.051 |
| | | Current | | | | 8 log |
| | | and two | | | | reduction |
| | | wire | | | | AC 0.051 |
| | | diameters | | | | 10 log |
| | | 0.051″ and | | | | reduction |
| | | 0.020″ | | | | |

We claim:

1. A catheter, comprising:
a body made of a biocompatible material and comprising silver-containing electrodes;
a power source having a first terminal and a second terminal with one of the terminals being in electrical communication with the silver-containing electrodes;
an impedance device placed in a current path between the first terminal of the power source and the second terminal of the power source reducing or inhibiting current flowing from the first terminal from reaching the second terminal; and
a current generator connected to the power source that creates a flow of antimicrobial ions from the silver-containing electrode when current passes through the silver-containing electrode.

2. A catheter of claim 1, further comprising electronics connected to the current generator to alternate cycling of the current from positive to negative and back to positive.

3. The medical device recited by claim 2, wherein the current along the silver-containing electrodes and the body tissue.

4. A method for inhibiting microbial infection associated with an indwelling catheter, comprising:
providing a catheter according to claim 1;
delivering a current to the silver-containing electrodes when the catheter is located in a human body at a site of potential infection, and wherein the delivery of current to the silver-containing electrodes causes release of an effective amount of silver metal ions to inhibit the microbial infection.

5. The method of claim 4, wherein the microbial infection comprises a Gram positive bacterium.

6. The method of claim 4, wherein the microbial infection comprises an antibiotic-resistant bacteria.

7. The method of claim 4, wherein the microbial infection comprises a Gram negative.

8. The method of claim 4, wherein the microbial infection comprise a fungus.

9. The method of claim 4, wherein the current is applied in a duty cycle that includes at least two current magnitudes, at least two current directions, and at least two current durations.

10. The method of claim 4, wherein the current is applied in a duty cycle that includes at least four current magnitudes, at least two current directions, and at least four current durations.

11. The method of claim 4, wherein the current is applied in 1-8 cycle periods.

12. The method of claim 9 wherein the cycle is:
a) +60 μA for 1 min
b) all off (0 μA) for 2 min
c) −60 μA for 1 min
d) all off (0 μA) for 2 min.

13. The method of claim 12, wherein the cycle is repeated 1-8 times.
